(19) United States
(12) Patent Application Publication
(10) Pub. No.: US 2005/0209588 A1
(43) Pub. Date: Sep. 22, 2005

(54) HIFU resculpturing and remodeling of heart valves

(75) Inventors: Eugene A. Larson, Lummi Island, WA (US); Perry W. Kaminski, Stehekin, WA (US)

Correspondence Address: ROBERT L. MCDOWELL, 1170 JACKSON HEIGHTS DR, WEBSTER, NY, 14580-9367, US

(73) Assignee: Crum, Kaminski & Larson, LLC

(21) Appl. No.: 11/130,771

(22) Filed: May 17, 2005

Related U.S. Application Data
(63) Continuation-in-part of application No. 10/921,715, filed on Aug. 19, 2004.
(60) Provisional application No. 60/560,089, filed on Apr. 7, 2004.
(60) Provisional application No. 60/500,067, filed on Sep. 4, 2003.
(60) Provisional application No. 60/644,671, filed on Jan. 18, 2005.

(51) Int. Cl. A61F 7/00; A61B 18/04; A61F 7/12

(52) U.S. Cl. 606/27; 607/96

(57) ABSTRACT

Resculpturing or altering the shape of cardiac valve leaflets, or cusps, and/or the valve supporting structures (such as the orifice or annulus, the aortic root or aorta, or the tendons which are attached to the valve) using heat. More specifically, the invention relates to applying High Intensity Focused Ultrasound (HIFU) during cardiac surgery to raise the temperature of heart valve structures comprised of or including collagen to a temperature sufficient to induce collagen change and shrinkage. The HIFU energy source (transducer) is preferably placed close to the valve structure during open heart surgery.

This application is a Continuation-in-Part of application Ser. No. 10/921,715 filed Aug. 19, 2004 which claims the benefit of U.S. Provisional Patent Application No. 60/560,089 filed Apr. 7, 2004 and U.S. Provisional Patent Application No. 60/500,067 filed Sep. 4, 2003.

This application claims the benefit of U.S. Provisional Patent Application No. 60/644,671 filed Jan. 18, 2005.

### FIELD OF THE INVENTION

The present invention is directed to resculpturing or altering the shape of cardiac valve leaflets, or cusps, and/or the valve supporting structures using heat, in particular High Intensity Focused Ultrasound (HIFU).

### BACKGROUND OF THE INVENTION

In 1999, a U.S. hospital discharge diagnosis of nonrheumatic mitral valve disease was made in 912,000 patients and nonrheumatic aortic valve disease in 519,000 patients. During that same year, there were 80,000 heart valve surgeries in the U.S.

Many patients with valvular heart disease are either too ill, or not ill enough, for open chest heart valve surgery intended to replace or repair the valve.

Many patients with heart valve disease have regurgitation or leaking of the heart valve as the predominant mode of native valve failure. 15 to 20% of patients with a bicuspid aortic valve (occurring in 2-6% of the population) and patients with mitral valve prolapse have valvular regurgitation, part of this patient group may be treated by surgically repairing the failing valve. The surgical repair techniques include procedures for removing or “gathering in a tuck” a portion of the excess tissue in the valve leaflet, resulting in an improved seal and reduced regurgitation.

Collagens, including collagenous bundles, fibers or collagenous lamina fibrosa are abundant in the valve leaflet. With reference to the aortic valve, the aortic orifice containing the aortic root and the sinuses of Valsalva are largely collagenous near the annulus.

Medical equipment and procedures have been developed to noninvasively cause physical dimension changes, such as shortening and thickening of collagen structures. The use of thermal energy to shrink redundant collagenous tissue, especially joint ligaments, has become widespread in orthopedic surgery. Other examples in medical practice include the treatment of collagen structure beneath the skin with heat produced noninvasively from RF energy applied to the surface of the skin. Shortening the collagen in this example ‘plumps up” and lessens the depth and appearance of facial wrinkles. Changes in collagens resulting in shrinkage begin to occur beyond about 50° C. by a combination of the fracture of intramolecular hydrogen bonds which results in the unraveling of the triple helix with the intermolecular bonds remaining intact.

U.S. Pat. Nos. 5,755,753; 6,453,202; 6,405,090 and 6,381,498, all to Knowlton, describe collagen denaturization by heat with subsequent shrinkage of the tissue in applications relating to the skin and aesthetic dermatology. U.S. Pat. Nos. 4,881,543 to Trembly et al. and 4,976,709 to Sand describe, respectively, microwave and laser induced heat to shrink collagen in the eye. Orthopedic applications, such as the thermal shrinkage of ligaments with RF (Radio Frequency) energy, are described in U.S. Pat. No. 5,458,596 to Lax et al.

United States Patent Application Publication No. 2003/0114901 to Loeb et al. describes shrinking chordae tendineae utilizing a catheter device which delivers thermal energy in contact with, or in close proximity to, a predetermined region of the chordae tendineae. Although ultrasound is named as one source of thermal energy, it is not taught how this energy can be precisely applied to the target tissue.

U.S. Pat. No. 6,355,030 to Aldrich et al. teaches a heating device, preferably a RF device, placed in direct contact with, or spaced a small distance from, a valve structure to cause thermal energy from the device to be thermally conducted to the structure to cause a degree of collagen shrinkage. Precise temperature control is critical to achieve the proper degree of shrinkage of the collagen fibers. As disclosed by Aldrich et al., temperature control is inferred from a temperature probe for measurement of the heating device itself or situated at the tissue site. In practice, because a valve leaflet is so thin and of such a low thermal mass a temperature probe would not respond quickly enough to provide precise temperature control within the intended target. The problem of determining the precise amount of energy delivered to the target from a radiating source such as RF is that the energy spreads from the source by the square of the distance adding difficulty and complexity to the task of monitoring the relative distance between the energy source, target and temperature probe. Furthermore, it is disclosed to place the heating device of U.S. Pat. No. 6,355,030 in direct contact with a valve structure or spaced a small distance from a valve structure by a fluid barrier of an electrolytic or thermally conductive fluid. While direct or nearly direct contact is essential for an RF energy source, such positioning is not appropriate for other energy sources, such as ultrasound, since a focal distance (acoustic standoff) is required to focus ultrasound energy from the full face diameter of an ultrasound transducer to a spot where the ultrasound focuses on the target tissue (see 5 in FIG. 1, for example).

U.S. Patent Application Publication No. 2003/0018358 to Saadat teaches the placement of a mechanical clip and/or sutures or wires into or on the valve leaflets or supporting structure via a catheter. It is a mechanical solution regarding the leaflets. The annulus and valve leaflet support structure may be heated by expanding a balloon on the annulus and delivering RF energy to electrodes on the Balloon, or using laser energy delivered by a fiberoptic. Saadat is concerned with shrinking the annulus of the valve or the supporting structures to the leaflets, not the actual leaflets themselves. Furthermore, Saadat cannot access a narrow portion of the leaflet to apply heat by his balloon method of delivering RF or Laser.

Collagen is the most abundant protein in the body, with type I collagen the most common in ligaments, tendons and heart valves. Type I collagen is formed by three protein chains which are wound together to form a triple helical structure. The crosslinking of the collagen molecules gives the structure a high tensile strength and stiffness. In its normal state collagen is ‘extended’ in rod-like fibrils. When heat is applied, the hydrogen bonds that hold the triple helix together are broken but the strong intermolecular bonds remain intact. The collagen collapses into random coils and the tissue shrinks lengthwise during this denaturization of the collagen. In human tissue it appears that optimal ‘shrinkage’ occurs between 65° and 70° Celsius. Collagen begins morphological retraction of fibrillation above approximately 50° C.

Collagen is an elongated protein that forms extremely strong but very small fibrils. Collagen fibrils are formed from long collagen molecules which are staggered in arrangement but tightly bound laterally by covalent chemical bonds. Collagen is a high molecular weight protein formed from three polypeptide strands twisted into a triple helix. Each strand is a left-handed helix twisted on itself, but the three strands are twisted into a larger right-handed triple helix. The triple helix is responsible for the stability of the molecule and for the property of self-assembly of molecules into microfibrils. The flexible parts of each strand projecting beyond the triple helix (telopeptides) are responsible for the bonding between adjacent molecules. The cross links that bind collagen molecules together laterally are made between the helical shaft of one molecule and the non-helical extension of an adjacent molecule.

Within an individual collagen molecule, the three polypeptide strands are linked together by stable intramolecular hydrogen bonds. The great strength of collagen fibers, however, originates mainly from the stable intermolecular covalent bonds between adjacent collagen molecules. Upon heating, the intramolecular hydrogen bonds break but the covalent bonds between molecules remain (FIGS. 2A, 2B). As the collagenous tissue is heated above about 50° C. intramolecular hydrogen bonds (a) break and the collagen triple helix unravels while intermolecular bonds (b) remain intact thereby resulting in the collagen chain structure shortening and thickening.

### SUMMARY OF THE INVENTION

The present invention is directed to resculpturing or altering the shape of cardiac valve leaflets, or cusps, and/or the valve supporting structures (such as the orifice or annulus, the aortic root or aorta, or the tendons which are attached to the valve) using heat. More specifically, this invention relates to applying High Intensity Focused Ultrasound (HIFU) during cardiac surgery to raise the temperature of heart valve structures comprised of or including collagen to a temperature sufficient to induce collagen change and shrinkage. The HIFU energy source (transducer) is preferably placed close to the valve structure during open heart surgery.

### BRIEF DESCRIPTION OF THE DRAWINGS

FIG. 1 shows a cross-section of a preferred embodiment of an ultrasound probe showing a remote HIFU energy source and an acoustic standoff.

FIGS. 2A and 2B illustrate the change in length (shortening) and thickening of collagen fibers within a section or all of a heart valve leaflet or the supporting structures, due to the controlled application of HIFU.

FIGS. 3A and 3B show a pig heart mitral valve leaflet before and after, respectively, the application of HIFU to induce collagen shrinkage.

FIG. 4 shows a lesion produced intraoperatively in the posterior wall of an animal heart.

### DETAILED DESCRIPTION OF THE PREFERRED EMBODIMENTS

In commonly assigned and copending U.S. patent application Ser. No. 10/921,715 filed Aug. 19, 2004 entitled “Treatment of Cardiac Arrhythmia Utilizing Ultrasound”, the disclosure of which is hereby incorporated by reference, a method is disclosed for treating arrhythmias of the heart utilizing continuous wave (CW) HIFU. It has now been discovered that cardiac valve disease and dysfunction may also be treated by methods incorporating HIFU.

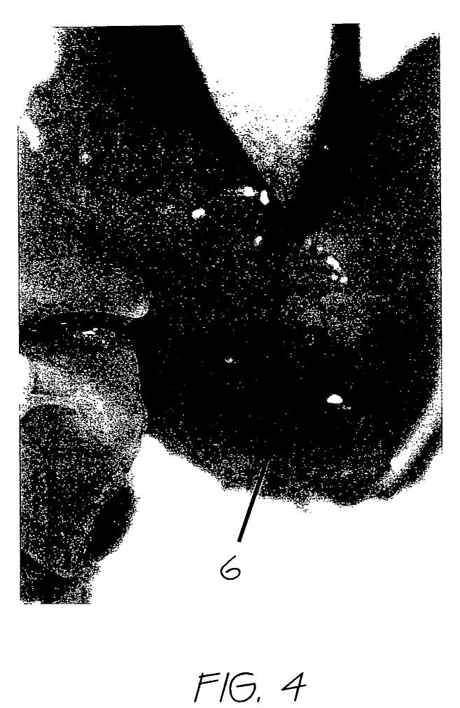

An in vivo animal experiment disclosed in parent U.S. application Ser. No. 10/921,715 demonstrates the effectiveness of producing a thermal effect using High Intensity Focused Ultrasound (HIFU) in a live pig heart. See FIG. 4. A lesion 6 in the endocardium of the posterior left ventricular wall was achieved by applying HIFU intraoperatively through the heart from the epicardial surface of the anterior left ventricular wall. The unfocused HIFU energy passed first through the anterior myocardium of the left ventricle, then through the blood-filled ventricular chamber to reach the endocardium of the posterior left ventricular wall where the HIFU power was focused. Tissue within the focal region, where the spatial peak intensity was greatest, was heated due to absorbed energy creating a lesion.

For this study, a HIFU system was utilized with total forward electrical power set to 60 watts. A HIFU transducer was selected with 4 MHz center frequency and a 5 cm fixed focal length. Because the region of interest in the myocardium was less than 5 cm from the front face of the transducer a truncated hydrogel cone was placed between the transducer and the epicardium to serve as an acoustic standoff. Hydrogel was chosen as the acoustic coupling path within the standoff because it is easy to handle and it is relatively unattenuating to the unfocused ultrasound energy propagating through it.

The transducer with an acoustic standoff was placed on the anterior left ventricular wall of the beating heart and continuous wave (CW) acoustic power applied in a single burst of ten seconds. Ultrasound energy generated within the transducer passed through the hydrogel, the anterior wall of the heart, the blood-filled ventricle, and focused on the endocardium of back wall of the left ventricle.

A lesion on the posterior ventricular myocardium was successfully created using HIFU applied from the anterior wall through the left ventricular cavity to the posterior wall. The photograph in FIG. 4 shows the lesion 6 produced intraoperatively in the posterior wall with the transducer device placed on the epicardium of the anterior left ventricular wall. The transducer and the origin of the HIFU are to the right of this picture. HIFU energy passed through the anterior wall, the blood-filled ventricular chamber and focused on the endocardium of the opposite posterior left ventricular wall as indicated in this picture. Intervening tissue (the anterior wall) appeared undamaged.

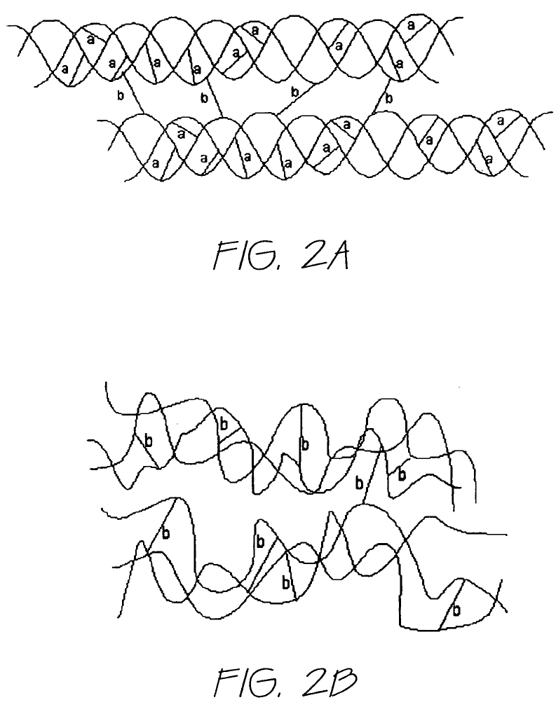

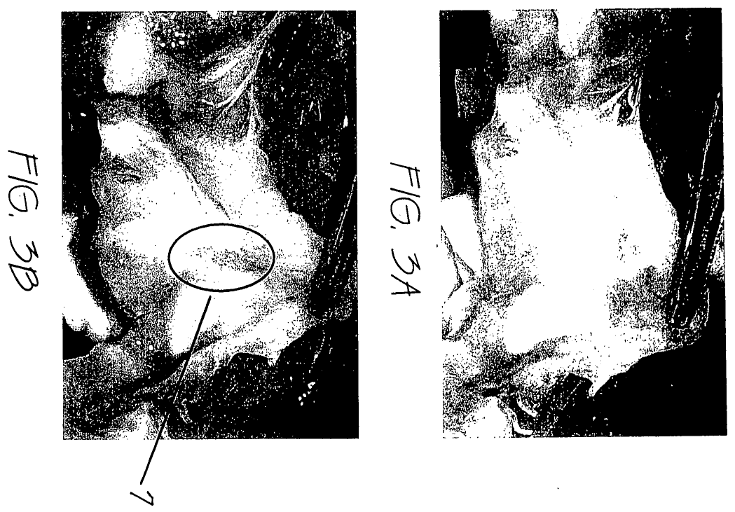

The present invention describes the creation of controlled remodeling or reshaping of cardiac valve structures by heating with HIFU and altering the configuration of collagen fibrils. At temperatures of about 50° to 60° C. collagen molecules begin to change shape and shrink (FIGS. 2A, 2B) and as a consequence, collagenous structures such as the valve leaflets, which are composed of primarily Type I collagen, will also show shrinkage (FIGS. 3A, 3B).

The preferred energy source is HIFU in a preferred frequency range of 1-15 MHz, and most preferably 5-10 MHz, to cause collagen deformation, typically shrinking and thickening, and thereby causing changes in size and shape of the collagenous structures of the heart valves. More specifically, the present invention relates to applying High Intensity Focused Ultrasound (HIFU) during cardiac surgery to raise the temperature of tissue comprised of or including collagen to a temperature sufficient to induce collagen denaturization and shrinkage (most preferably at approximately 55°-60° C.). HIFU thermal denaturization of collagen can be directed within a section of, or all of, a valve leaflet, chordae tendineae, annuli, aortic root or other valve related structures, so that collagen tissue will shorten and thicken as exemplified by FIG. 3B.

Thermal denaturization and shrinkage is an irreversible process beyond about 55° C. By selectively placing the HIFU heat zone, shortening and thickening action can change the shape of a valve leaflet or cusp. When applied to the valve annulus or orifice, aortic root or aorta, HIFU induced heat will cause the diameter to reduce and when applied to connecting valve tendons, will cause them to shorten. These changes in the cusp shape, diameter of the valve annulus or length of the connecting cusp tendons can be used to reduce or eliminate regurgitation of blood through the valve (improving the closed valves' hemodynamic seal.)

The inventors discovered that exposing a leaflet of the mitral valve to HIFU during cardiac surgery in a live porcine model resulted in a section of the leaflet changing shape due to shrinkage in the region of treatment. However, the HIFU did not perforate or destroy the leaflet. A 4.0 MHz transducer fitted with a 30 mm hydrogel acoustic delay line was used to create a short line of visibly shrunken collagen within a few seconds.

Additional experiments were carried out on ex vivo pig heart mitral valve leaflets (see FIG. 3B). In this example, a mitral valve leaflet was partially dissected from a whole heart of a 6 month pig that had been removed and chilled approximately 24 hours prior to experiment. The valve leaflet remained attached to the mitral valve annulus. A HIFU treatment line 7 approximately 1 cm long can be seen in the center of leaflet. Direction of the HIFU line 7 in FIG. 3B was from outer edge toward valve annulus (top-to-bottom in picture). Shrinkage is evident in the area of the HIFU treatment. No perforation and no apparent degradation of strength was observed.

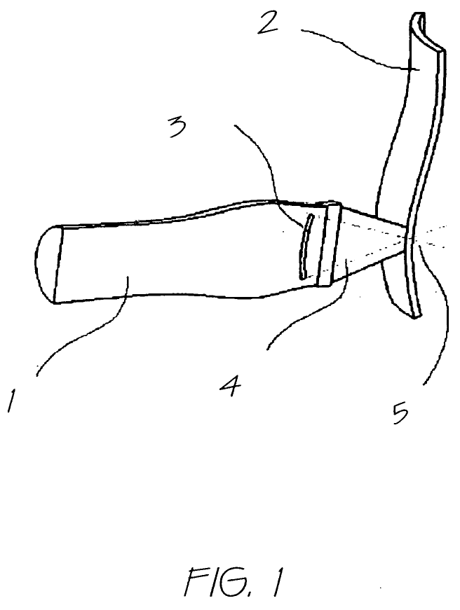

The inventive method comprises:
A. Visualizing the heart valve, including the leaflets and the supportive structure, attachments and the annulus, during open chest cardiac surgery;
B. Delivering HIFU energy to the valve leaflets, chordae tendineae, and/or the annuli to introduce a controlled amount of heating of the collagen to approximately 55° C. or beyond, thus causing structural change (contraction and thickening) in the leaflets, chordae tendineae, or annuli. In the present invention, it is most preferable that the energy source (HIFU) is delivered to the cardiac structure from a remote origin 3 within the ultrasound probe 1 (FIG. 1), through an ultrasound coupling medium or delay line 4 (energy source is not in direct contact with the cardiac valve structure). This coupling medium 4 (acoustic standoff) couples the remote energy source to a focal zone 5 on the valve structure 2 and can be in the form of a hydrogel or metal acoustic delay line or a water column; and,
C. Reducing or eliminating cardiac valvular regurgitation by the shortening of the chordae tendineae, changes in the shape of the valve leaflet and/or the reduction in diameter of the valve annulus resulting from the thermal shrinkage of collagen.

The present invention is based on observations during in vivo and ex vivo porcine heart surgery that components of the heart valve structure contracted when heated with High Intensity Focused Ultrasound. A specific example is shown in FIG. 3B where a porcine mitral valve leaflet was treated ex vivo, but still attached to the heart and annulus, with a HIFU transducer operating at a nominal frequency of 4.0 MHz for 10 seconds continuously at 30 Watts total power through a 30 mm hydrogel acoustic delay line. In this example, the distal tip of the hydrogel acoustic delay line was in direct contact with the mitral valve leaflet while the energy source (the piezoelectric element) was contained within the transducer housing separated from the tip by 30 mm of the hydrogel acoustic delay line.

The ex vivo experiment described above shows that the desired therapeutic effect can indeed be achieved by applying HIFU to a portion of a valve structure, in this example a mitral valve leaflet. For open heart surgery, a smaller version of the HIFU probe may be utilized to facilitate maneuvering the probe to the target valve component.

The hand held HIFU probe 1 (FIG. 1) of the inventive method is designed to be used during open chest cardiac surgery to contact a valve structure with an acoustic coupling element, such as a hydrogel, glass, metal or polymer standoff, or a column of water or blood, thus delivering HIFU generated remote from the treatment contact point and through the coupling member placing a thermal dose into the structure sufficient to raise the temperature to thermally shrink the included collagen.

As shown in FIG. 1, the HIFU probe 1 contains a piezoelectric transducer element 3 to provide the HIFU energy source that is positioned within the probe housing and is not in direct contact with the target tissue. Ultrasound energy from the piezoelectric element is focused toward, and emerges at or near, the distal tip of the probe so that acoustic energy of sufficient intensity (HIFU, or High Intensity Focused Ultrasound) occurs in a focal zone 5 which is placed precisely within the target tissue 2.

In the cross-sectional drawing of FIG. 1, the region of the probe within which focusing occurs is the acoustic standoff 4 and it is this component that may be in contact with the target tissue. Alternatively, the acoustic standoff 4 may be separated by a small layer or film of coupling fluid, such as blood or saline. In the preferred embodiment, the acoustic standoff is preferably a low attenuating material such as a silicone or hydrogel. The target tissue 2 shown is a section of a valve leaflet but other valve structures may also be the target tissue, such as supporting annuli and tendons.

The HIFU probe is attached via an electric cable assembly to a power source within the control module which generates an electric signal of the appropriate frequency to drive the piezoelectric element at the desired ultrasound frequency. In this mode, the ultrasound probe is a transmitter, creating ultrasound energy which propagates into the target tissue. Also, while delivery of the ultrasound in a continuous wave (CW) manner is preferred, delivery in a pulsed or intermittent manner is also contemplated for the inventive method.

The ultrasound probe may also operate as a receiver of some of the reflected ultrasound energy and convert the ultrasound into an electric signal which is received by the electronic control module. This returning energy may be utilized in several important ways:
1. The returning ultrasound signal, the “echo”, typically develops at an interface between materials of different acoustic impedances and can be precisely measured in time relative to the transmitted signal. An echo, for example, will arrive from both anterior and posterior surfaces of a valve leaflet (or other targeted cardiac structure) and the timing information can by utilized to determine the thickness of the leaflet. This thickness information can then be used to adjust the power of the subsequent HIFU treatment of the leaflet.
2. As the HIFU treatment continues, collagen in the tissue will begin to change in structure and nature and will have changing acoustic properties. These changes, such as acoustic velocity, attenuation and impedance) can be detected and monitored and then utilized to determine the amount of denaturization that has occurred and hence the degree of desired collagen and tissue shrinkage.
3. The characteristics of the returning ultrasound signal may also be analyzed to infer the actual temperature of the target tissue.

It is believed that appropriate patients for the inventive method include those who are candidates for the Percutaneous Heart Valves developed by Edwards Lifesciences, Corevalve, Sanra Medical, Cardiomend and others. The first patients to receive the percutaneous valves will be those who are too sick to undergo open chest valve surgery and those patients not sufficiently sick to justify valve surgery. With this group of patients (estimated to be 300,000 per year), the inventive HIFU valve repair method could be attempted.

Another group of patients are those in which open chest heart surgery is performed in which it is desirable to attempt to repair a cardiac valve before deciding to replace it. This includes patients with multiple valve dysfunction where it is desirable to repair and restore the hemodynamic closure and seal of some valves while replacing others. Examples of this condition include the HIFU repair of a floppy mitral valve and the replacement of a stenotic aortic valve.

While the invention has been described with reference to preferred embodiments it is to be understood that the invention is not limited to the particulars thereof. The present invention is intended to include modifications which would be apparent to those skilled in the art to which the subject matter pertains.

1. A method for reducing cardiac valve regurgitation by inducing thermal changes to collagen tissue utilizing High Intensity Focused Ultrasound (HIFU), said method comprising:
direct optical visualization of a heart valve region of interest comprising cardiac valve tissue and/or support structure;
applying a HIFU source which is not in direct contact with the cardiac valve tissue and/or support structure;
emitting therapeutic ultrasound energy from said HIFU source;
focusing the HIFU energy on selected cardiac valve tissue and/or support structure to raise the temperature of the selected heart valve region of interest comprising collagen to a temperature sufficient to induce collagen change and shrinkage.

2. The method of claim 1 wherein the region of interest comprises a valve leaflet.

3. The method of claim 1 wherein the region of interest comprises a semilunar valve cusp.

4. The method of claim 1 wherein the region of interest comprises any of the chordae tendineae.

5. The method of claim 1 wherein the region of interest is a supporting valve annulus.

6. The method of claim 1 wherein the HIFU is delivered to the cardiac valve and/or support structure during an “open chest” surgical procedure by a hand held HIFU probe having the piezoelectric energy source separated from the tissue by an acoustic delay.

7. The method of claim 1 further comprising receiving reflected ultrasound energy and analyzing the reflected ultrasound energy to determine process characteristics of the selected heart valve region of interest.

8. The method of claim 7 wherein the detected and measured characteristics of the reflected ultrasound energy include at least one of acoustic velocity, impedance and/or attenuation.

9. The method of claim 7 wherein the process characteristics include at least one of thickness of the region of interest, amount of tissue shrinkage, and temperature of the region of interest.

10. An apparatus for use in reshaping the valve structures within a patient's heart, said apparatus comprising:
ultrasound emitting means having a surface adapted for placement directly on a valve structure by incorporating an acoustic delay line;
focusing means for focusing the emitted therapeutic ultrasound energy on said region of interest of said patient's heart;
wherein said level and/or frequency of said ultrasound energy is selected to induce collagen change and shrinkage to produce reshaping of a valve structure.

11. The apparatus of claim 9 wherein the acoustic delay line comprises an acoustic standoff.

12. The apparatus of claim 10 wherein the acoustic standoff comprises a hydrogel.

13. The apparatus of claim 10 wherein the acoustic standoff comprises a polymer.

14. The apparatus of claim 9 wherein said ultrasound energy comprises High Intensity Focused Ultrasound.

15. The apparatus of claim 12 having a frequency range of 1-15 MHz.

16. The apparatus of claim 9 wherein the emitted ultrasound is delivered in a continual manner.

17. The apparatus of claim 9 wherein the emitted ultrasound is delivered in a pulsed or intermittent manner.

18. The apparatus of claim 9 wherein the ultrasound emitting means comprises a probe containing a piezoelectric transducer element.

19. The apparatus of claim 15 wherein the probe further comprises a receiver for receiving reflected ultrasound energy.
